(19) United States
(12) Patent Application Publication
(10) Pub. No.: US 2024/0336564 A1
(43) Pub. Date: Oct. 10, 2024

(54) COMPOUND, RUBBER MIXTURE CONTAINING THE COMPOUND, VEHICLE TIRE WHICH HAS AT LEAST ONE COMPONENT COMPRISING THE RUBBER MIXTURE, PROCESS FOR PREPARING THE COMPOUND, AND USE OF THE COMPOUND AS AN AGING PROTECTION AGENT AND/OR ANTIOZONANT AND/OR COLORANT

(71) Applicant: CONTINENTAL REIFEN DEUTSCHLAND GMBH, Hannover (DE)

(72) Inventors: Andreas Jacob, Hannover (DE); David-Raphael Dauer, Dassel (DE); Julian Strohmeier, Einbeck (DE)

(73) Assignee: Continental Reifen Deutschland GmbH, Hannover, OT (DE)

(21) Appl. No.: 18/291,152

(22) PCT Filed: Jun. 14, 2022
(86) PCT No.: PCT/DE2022/200130

§ 371 Date: Jan. 22, 2024

(30) Foreign Application Priority Data
Jul. 23, 2021 (DE) 10 2021 207 926.1

(51) Int. Cl. C07D 209/14; B60C 1/00; C08K 5/3417; C09B 57/00

(52) U.S. Cl. CPC C07D 209/14; B60C 1/0016; B60C 1/0025; C08K 5/3417; C09B 57/00

(57) ABSTRACT

A compound, a rubber mixture containing the compound, a vehicle tire comprising the rubber mixture in at least one component, a process for producing the compound and the use of the compound as an aging stabilizer and/or antiozonant and/or dye, in which, the compound has the formula (I), wherein A is an aromatic radical optionally bearing additional substituents, and wherein R¹ is selected from the group consisting of xi) aromatic radicals, wherein the aromatic radicals optionally bear substituents selected from the group consisting of halogen radicals, cyano radicals, ester radicals, ketone radicals, ether radicals and thioether radicals, and xii) linear, branched and cyclic aliphatic C₁- to C₁₂-radicals, and xiii) combinations of aromatic and aliphatic C₁- to C₁₂-radicals.

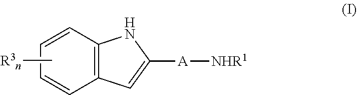

### CROSS-REFERENCE TO RELATED APPLICATIONS

The present application is a National Stage Application under 35 U.S.C. § 371 of International Patent Application No. PCT/DE2022/200130 filed on Jun. 14, 2022, which claims priority from German Patent Application No. 10 2021 207 926.1 filed on Jul. 23, 2021, the disclosures of which are herein incorporated by reference in their entireties.

### TECHNICAL FIELD

The invention relates to a compound, to a rubber mixture containing the compound, to a vehicle tire comprising the rubber mixture in at least one component, to a process for producing the compound and to the use of the compound as an aging stabilizer and/or antiozonant and/or dye.

### BACKGROUND

It is known that vehicle tires and technical rubber articles employ polymeric materials such as especially rubbers.

In case of prolonged storage and especially in the target application which is often at elevated temperatures, natural rubber and synthetic polymers (such as IR, BR, SSBR, ESBR etc.) but also natural and synthetic oils, fats and lubricants are subject to oxidation reactions which have an adverse effect on the originally desired properties. Depending on the type of the polymer the polymer chains are shortened right up to the liquefaction of the material or subsequent hardening of the material occurs.

Aging stabilizers thus play a decisive role in the durability of vehicle tires and other technical rubber articles.

Known aging stabilizers are aromatic amines, for example
6-PPD (N-(1,3-dimethylbutyl)-N′-phenyl-p-phenylenediamine),
IPPD (N-isopropyl-N′-phenyl-p-phenylenediamine) or
SPPD (N-(1-phenylethyl)-N′-phenyl-p-phenylenediamine).

These molecules can react with oxygen or ozone or free radicals formed, such as alkyl, alkoxy and alkylperoxy radicals, and thus scavenge these and accordingly protect the rubbers etc. from further oxidation reactions.

However, a disadvantage of this substance class is that they are suspected to be carcinogenic.

Aging stabilizers which especially react with ozone and effect scavenging thereof are also referred to as “antiozonants”.

### SUMMARY

It is an object of the invention to provide a novel compound which can especially be used as an aging stabilizer in vehicle tires or other technical rubber articles, specifically with a lower hazard potential coupled with sufficient solubility in the respective matrix, for example and in particular in the polymer. This is intended to ensure continuing optimal protection from oxygen and ozone and to prevent the tendency for blooming while reducing hazardousness to health.

The object is achieved by a compound having the general formula I):

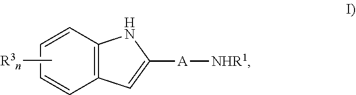

wherein A is an aromatic radical optionally bearing additional substituents, and wherein R¹ is selected from the group consisting of
xi) aromatic radicals, wherein the aromatic radicals optionally bear substituents selected from the group consisting of halogen radicals, cyano radicals, ester radicals, ketone radicals, ether radicals and thioether radicals, and xii) linear, branched and cyclic aliphatic C₁- to C₁₂-radicals and xiii) combinations of aromatic and aliphatic C₁- to C₁₂-radicals,
and wherein R³ is selected from the group consisting of linear, branched and cyclic, saturated and unsaturated, aliphatic C₁- to C₁₂-radicals optionally bearing one or more halogen substituents, aryl radicals optionally bearing one or more halogen substituents and halogen radicals, wherein fluorine, bromine and chlorine are preferred, cyano radicals, ester radicals, ketone radicals, ether radicals and thioether radicals and wherein n assumes the value 0 or 1 or 2 or 3 or 4, wherein when n is 2 or 3 or 4 the radicals R³ are independently of one another identical or different.

The object also is achieved by the inventive rubber mixture containing the compound and also by the inventive vehicle tire comprising the inventive rubber mixture in at least one component.

The object is further achieved by using the compound as an aging stabilizer and/or antiozonant.

The compound may further be used as a dye.

The object is further achieved by the process according to the invention for producing the compound according to the invention.

### DETAILED DESCRIPTION

The present disclosure provides a compound having the general formula I):

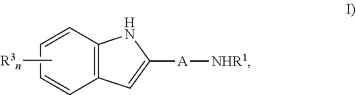

wherein A is an aromatic radical optionally bearing additional substituents, and wherein R¹ is selected from the group consisting of
xi) aromatic radicals, wherein the aromatic radicals optionally bear substituents selected from the group consisting of halogen radicals, cyano radicals, ester radicals, ketone radicals, ether radicals and thioether radicals,
and xii) linear, branched and cyclic aliphatic C₁- to C₁₂-radicals and xiii) combinations of aromatic and aliphatic C₁- to C₁₂-radicals,
and wherein R³ is selected from the group consisting of linear, branched and cyclic, saturated and unsaturated, aliphatic C₁- to C₁₂-radicals optionally bearing one or more halogen substituents, aryl radicals optionally bearing one or more halogen substituents and halogen radicals, wherein fluorine, bromine and chlorine are preferred, cyano radicals, ester radicals, ketone radicals, ether radicals and thioether radicals and wherein n assumes the value 0 or 1 or 2 or 3 or 4, wherein when n is 2 or 3 or 4 the radicals R³ are independently of one another identical or different.

It is preferable when A is an aromatic radical optionally bearing additional substituents, and
wherein R¹ is selected from the group consisting of benzyl and linear, branched and cyclic aliphatic C₁- to C₁₂-radicals, and
wherein R³ is selected from the group consisting of linear, branched and cyclic aliphatic C₁- to C₁₂-radicals, and aryl radicals, cyano radicals, halogen radicals, wherein fluorine, bromine and chlorine are preferred, ether radicals and thioether radicals, and wherein n assumes the value 0 or 1 or 2 or 3 or 4, wherein when n is 2 or 3 or 4 the radicals R³ are independently of one another identical or different.

It is apparent to those skilled in the art that when n is 0 or 1 or 2 or 3 all remaining free positions at the benzene ring of the indole structure are hydrogen atoms.

It is likewise apparent to those skilled in the art that the representation of the bond of (R³)ₙ to the benzene ring of the indole structure is to be understood as meaning that these group(s) may each be arranged at any position on the benzene ring except, naturally, two or more (R³) simultaneously at the same position as would already be ruled out by the tetravalent nature of the carbon atom of the benzene ring.

In the context of the present invention descriptions of the kind “C₁- to C₁₂-radicals” are to be understood as meaning radicals having 1 to 12 carbon atoms. Irrespective of this “C₁” is also used to describe the position of the most highly oxidized carbon atom/the highest priority according to the Cahn-Ingold-Prelog convention (CIP). It is apparent to those skilled in the art what is meant in the respective context.

The compound according to the invention is an indole derivative and exhibits a lower hazard potential relative to known aging stabilizers based on aniline (possible cleavage product of 6-PPD). Comparing the safety data sheets of the basic structures aniline and indole reveals that, unlike aniline, indole is neither genotoxic nor mutagenic. This is a crucial advantage, especially in a technical application such as in vehicle tires or other rubber products, since the rubber ingredients can be liberated by abrasion or other degradation processes. In addition, the oxidation products of the 6-PPD pose a particular risk to Coho salmon. It should therefore be assumed that this applies to aquatic organisms in general (Tian et al, Science, 2020 Z. Tian, Science, 2021, 371 (6525), 185-189).

Indole derivatives by contrast are proposed in pharmaceutical compositions or compositions for skin care as disclosed in US 20200339581 A1 and JP 2004196699A.

JP06147585B2 discloses indole derivatives of formula S1)

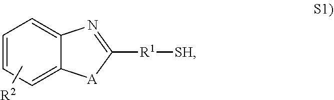

wherein in JP06147585B2 R¹ and R² are defined differently from the present case.

Compared to indole derivatives from the prior art as shown in formula S1) the compound according to the invention has the advantage that it comprises no vulcanizable groups (such as —SH) that allow bonding to rubber/polymers. Through bonding, the molecules would be locally bound and thus might not be effective at a remote location where oxidative stress occurs. Bonding would thus prevent the molecules from manifesting their full protection as aging stabilizers and/or antiozonants.

The invention comprises all advantageous embodiments which are reflected inter alia in the claims. The invention especially also comprises embodiments which result from a combination of different features with different levels of preference for these features so that the invention also comprises a combination of a first feature described as “preferred” or described in the context of an advantageous embodiment with a further feature described for example as “particularly preferred”.

It is preferable when A is selected from the group consisting of phenylene, naphthylene and antracenylene radicals, and phenylene, naphthylene and anthracenylene radicals having one or more substituents, wherein the substituents are preferably selected from the group consisting of linear, branched and cyclic aliphatic C₁- to C₁₂-radicals, and aryl radicals, cyano radicals, halogen radicals, wherein fluorine, bromine and chlorine are preferred, ether radicals and thioether radicals.

It is particularly preferable when A is a phenylene radical and thus a phenyl radical having two or more bonded substituents. This results in a particularly advantageous solubility of these compounds according to the invention in rubber mixtures, in particular for vehicle tires and other technical rubber articles.

The indole radical and the group NHR¹ at the benzene ring (of the phenylene radical) are preferably arranged para to one another.

It is preferable if the compound according to the invention has the structure of formula II):

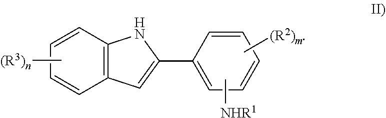

wherein R¹ is selected from the group consisting of
xi) aromatic radicals, wherein the aromatic radicals optionally bear substituents selected from the group consisting of halogen radicals, cyano radicals, ester radicals, ketone radicals, ether radicals and thioether radicals,
and xii) linear, branched and cyclic aliphatic C₁- to C₁₂-radicals,
and xiii) combinations of aromatic and aliphatic C₁- to C₁₂-radicals, and wherein R² is selected from the group consisting of linear, branched and cyclic, saturated and unsaturated, aliphatic C₁- to C₁₂-radicals optionally bearing one or more halogen substituents, aryl radicals optionally bearing one or more halogen substituents and halogen radicals, wherein fluorine, bromine and chlorine are preferred, cyano radicals, ester radicals, ketone radicals, ether radicals and thioether radicals, and
wherein m assumes the value 0 or 1 or 2 or 3 or 4, wherein when m is 2 or 3 or 4 the radicals R² are independently of one another identical or different, and
wherein R³ is selected from the group consisting of linear, branched and cyclic, saturated and unsaturated, aliphatic C₁- to C₁₂-radicals optionally bearing one or more halogen substituents, aryl radicals optionally bearing one or more halogen substituents and halogen radicals, wherein fluorine, bromine and chlorine are preferred, cyano radicals, ester radicals, ketone radicals, ether radicals and thioether radicals and wherein n assumes the value 0 or 1 or 2 or 3 or 4, wherein when n is 2 or 3 or 4 the radicals R³ are independently of one another identical or different.

Here too then when n is 0 or 1 or 2 or 4 the free positions at the benzene ring are hydrogen atoms.

It is likewise apparent to those skilled in the art that the representation of the bonds of (R²)ₘ and R¹HN to the benzene ring is to be understood as meaning that these groups may each be arranged at any position on the benzene ring except, naturally, not both simultaneously at the same position as would already be ruled out by the tetravalent nature of the carbon atom of the benzene ring.

It is preferable when n is 0 which applies to formula I) and II).

The radical R¹ is selected from the group consisting of xi) aromatic radicals, wherein the aromatic radicals optionally bear substituents selected from the group consisting of halogen radicals, cyano radicals, ester radicals, ketone radicals, ether radicals and thioether radicals, and xii) linear, branched and cyclic aliphatic C₁- to C₁₂-radicals and xiii) combinations of aromatic and aliphatic C₁- to C₁₂-radicals.

The aromatic radical from subgroup xi) is for example and preferably a phenyl radical.

The aromatic radicals of subgroup xi) may bear substituents.

As mentioned above these are selected from the group consisting of halogen radicals, cyano radicals, ester radicals, ketone radicals, ether radicals and thioether radicals.

It is preferable when the substituents are selected from the group consisting of ester radicals, ketone radicals, ether radicals and thioether radicals.

In preferred embodiments the aromatic radical is not substituted at the two carbon atoms adjacent to the C₁ atom, i.e. the carbon atom bonded to the N atom. In the case of a benzene ring as the basic structure it is thus preferable when there is no substituent in the ortho-position to the N atom.

In further preferred embodiments the aromatic radical of subgroup xi) is not substituted.

It is preferable when R¹ is bonded to the nitrogen atom (N) via a tertiary carbon atom. The C₁ atom is thus preferably a tertiary carbon atom.

In the context of the present invention the term “tertiary carbon atom” is to be understood as meaning a carbon atom which is bonded to only one hydrogen atom.

This results—relative to secondary and quaternary carbon atoms—in a particularly good protective effect due to the presence of the compound in rubber mixtures, in particular for vehicle tires and other technical rubber articles, and thus in particular in optimal reactivity in connection with the mechanisms relevant for aging stabilization, wherein undesired side reactions are avoided.

The mixed aromatic and aliphatic radical of subgroup xiii) is for example and preferably selected from the group consisting of benzyl and 1-phenylalkyl radicals having altogether 7 to 18 carbon atoms, in particular selected from benzyl and 1-phenylethyl radicals, wherein 1-phenylalkyl radicals, in particular 1-phenylethyl, are particularly preferred on account of the tertiary carbon atom.

It is particularly preferable when R¹ is a branched alkyl radical having 3 to 12 carbon atoms, in turn preferably 3 to 8 carbon atoms. It is preferable when at least one branching is present on the C₁ atom, i.e. on the carbon atom which is bonded to the nitrogen atom (N), thus making the C₁ atom a tertiary carbon atom.

It is very particularly preferable when R¹ is selected from 1,3-dimethylbutyl and cyclohexyl radicals and it is in turn very particularly preferable when R¹ is a 1,3-dimethylbutyl radical.

The radical/the radicals R² are independently of one another identical or different and are selected from the group consisting of linear, branched and cyclic, saturated and unsaturated, aliphatic C₁- to C₁₂-radicals optionally bearing one or more halogen substituents, aryl radicals optionally bearing one or more halogen substituents, and halogen radicals, wherein fluorine, bromine and chlorine are preferred, cyano radicals, ester radicals, ketone radicals, ether radicals and thioether radicals.

The recited radicals R² may in particular already be bonded to the respective benzene ring/the precursor thereof through selection of suitable starting substances.

It is preferable when m in formula II) is 0 (zero).

The indole radical and the group NHR¹ on the benzene ring (of the phenylene radical) are preferably arranged para to one another.

In a preferred embodiment the compound has the structure of formula III):

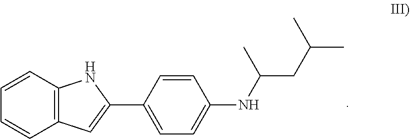 [SMILES: CC(C)CC(C)Nc1ccc(-c2cc3ccccc3[nH]2)cc1]

The compound of formula III) makes it possible to achieve optimal protection against oxidation and thus aging, in particular in polymers. At the same time, the compound of formula III) is markedly less hazardous to health than for example 6-PPD or other representatives of this substance class, as mentioned above.

The inventive compound according to formula I), formula II), formula III) and all of the foregoing is particularly suitable as an aging stabilizer and/or antiozonant in vehicle tires and/or technical rubber articles, such as in particular an air spring, bellows, conveyor belt, belt, drive belt, hose, rubber band, profile, a seal, a membrane, tactile sensors for medical applications or robotics applications, or a shoe sole or parts thereof and/or oils and/or lubricants.

The present invention therefore further provides for the use of the compound according to the invention as an aging stabilizer and/or antiozonant in vehicle tires and/or technical rubber articles, such as in particular an air spring, bellows, conveyor belt, belt, drive belt, hose, rubber band, profile, a seal, a membrane, tactile sensors for medical applications or robotics applications, or a shoe sole or parts thereof and/or oils and/or lubricants.

To use the compound of formula I), formula II), formula III) and all of the foregoing in the recited articles or substances, said compound is used in a composition and employed incorporated in said composition.

In vehicle tires or other technical rubber articles said composition is in particular a rubber mixture.

The present invention further provides for the use of the inventive compound of formula I), formula II), formula III) and all of the foregoing as a dye in fibers and/or polymers and/or paper and/or in (decorating) paints and coatings.

A further aspect of the present invention is a process for producing the compound of formula I) which comprises the following process steps:
a) providing the compound of formula A)

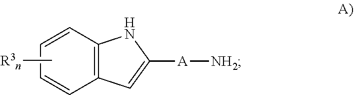

b) reacting the compound of formula A) with hydrogen or a hydrogenating reagent, in particular a hydride, and a ketone or aldehyde (R¹═O), preferably ketone, in particular and preferably methyl isobutyl ketone, to afford the compound of formula I)

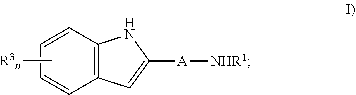

All of the foregoing applies to the radicals R¹ and R³ and n and A. Here too it is preferable when n is zero.

A “hydrogenation reagent” is to be understood as meaning a compound that effects hydrogenation. Such reagents include hydrides, in particular metal hydrides, as is known to those skilled in the art.

Suitable hydrides include for example sodium borohydride.

In the context of the present invention hydrogen is not additionally listed under “hydrogenation reagent” since it is explicitly mentioned as an alternative. Nevertheless it will be appreciated that the term “hydrogenation reagent” encompasses all reagents that form hydrogen, which effects the hydrogenation, in situ.

It is preferable when the reaction in step b) with hydrogen (H₂) and the ketone or aldehyde, preferably ketone, is carried out using a hydrogenation catalyst and preferably at a temperature of 50° C. to 70° C., in particular 60° C. for example. The reaction mixture is preferably subjected to hydrogen at a pressure of 15 to 25 bar, in particular 20 bar for example, and preferably subsequently stirred for 1 to 20 hours, preferably 8 to 13 hours, in particular 10 hours for example.

The ketone in step b) is the ketone derivative of the subsequent radical R¹; in the case of an aldehyde it is accordingly the aldehyde derivative.

For the sake of simplicity the abbreviated formula R¹═O is used for the aldehyde or ketone since the radical R¹ is the part that remains on the nitrogen atom after the reaction with the aldehyde or ketone.

It is preferable to employ the ketone methyl isobutyl ketone.

It is preferable when the process steps in which a reaction with hydrogen is carried out employ a suitable catalyst, referred to in the context of the present invention as “hydrogenation catalyst”.

It is preferable when the hydrogenation catalyst of the process is a noble metal catalyst, such as in particular palladium (Pd) or platinum (Pt). It is preferable when the noble metal is employed on carbon (C), such as palladium on carbon (Pd/C).

It is further also possible to employ other known catalysts, such as Raney nickel or copper chromite.

It is preferable when the reaction with hydrogen in step b) is carried out in a vessel suitable for the relatively high pressures, such as especially an autoclave or another pressure reactor.

In the abovementioned process the A is preferably a phenylene radical and m is zero. The bonds on the benzene ring (of the phenylene radical) are preferably para to one another.

As mentioned above the present invention further provides a rubber mixture.

The rubber mixture according to the invention contains the compound of formula I), in particular of formula II), in particular of formula III). The rubber mixture according to the invention may in principle be any rubber mixture in which in particular the novel inventive compound of formula I), in particular of formula II), in particular of formula III), acts as an aging stabilizer and/or antiozonant at low toxicity.

The rubber mixture of the invention contains at least one rubber.

It is preferable when the rubber mixture according to the invention contains 0.1 to 10 phr, particularly preferably 0.1 to 7 phr, very particularly preferably 1 to 6 phr, of the compound of formula I), in particular of formula II), in particular of formula III).

The unit “phr” (parts per hundred parts of rubber by weight) used in this document is the conventional indication of quantity for mixture recipes in the rubber industry. The dosage of the parts by weight of the individual substances is based in this document on 100 parts by weight of the total mass of all high molecular weight (M_{W} greater than 20 000 g/mol) rubbers present in the mixture.

In advantageous embodiments of the invention the rubber mixture according to the invention contains at least one diene rubber.

The rubber mixture may accordingly contain a diene rubber or a mixture of two or more different diene rubbers.

Diene rubbers are rubbers which are formed by polymerization or copolymerization of dienes and/or cycloalkenes and thus have C═C double bonds either in the main chain or in the side groups.

The diene rubber is preferably selected from the group consisting of natural polyisoprene (NR), synthetic polyisoprene (IR), epoxidized polyisoprene (ENR), butadiene rubber (BR), butadiene-isoprene rubber, solution-polymerized styrene-butadiene rubber (SSBR), emulsion-polymerized styrene-butadiene rubber (ESBR), styrene-isoprene rubber, liquid rubbers having a molecular weight M_{W} of more than 20 000 g/mol, halobutyl rubber, polynorbornene, isoprene-isobutylene copolymer, ethylene-propylene-diene rubber, nitrile rubber, chloroprene rubber, acrylate rubber, fluororubber, silicone rubber, polysulfide rubber, epichlorohydrin rubber, styrene-isoprene-butadiene terpolymer, hydrogenated acrylonitrile butadiene rubber and hydrogenated styrene-butadiene rubber.

Nitrile rubber, hydrogenated acrylonitrile-butadiene rubber, chloroprene rubber, butyl rubber, halobutyl rubber and/or ethylene-propylene-diene rubber in particular are used in the production of industrial rubber articles, such as belts, drive belts and hoses, and/or footwear soles. The mixture compositions known to those skilled in the art for these rubbers, which are specific in terms of fillers, plasticizers, vulcanization systems and additives, are preferably employed.

The natural and/or synthetic polyisoprene of all embodiments may be either cis-1,4-polyisoprene or 3,4-polyisoprene. However, the use of cis-1,4-polyisoprenes having a cis-1,4 proportion of >90% by weight is preferred. Such a polyisoprene is firstly obtainable by stereospecific polymerization in solution with Ziegler-Natta catalysts or using finely divided lithium alkyls. Secondly, natural rubber (NR) is one such cis-1,4-polyisoprene, for which the cis-1,4 content in the natural rubber is greater than 99% by weight.

A mixture of one or more natural polyisoprenes with one or more synthetic polyisoprenes is further also conceivable.

In the context of the present invention the term “natural rubber” is to be understood as meaning naturally occurring rubber which may be obtained from Hevea rubber trees and from “non-Hevea” sources. Non-Hevea sources include for example guayule shrubs and dandelion such as for example TKS (Taraxacum kok-saghyz; Russian dandelion).

If the rubber mixture of the invention contains butadiene rubber (i.e. BR, polybutadiene), this may be any of the types known to those skilled in the art. These include what are called the high-cis and low-cis types, with polybutadiene having a cis content of not less than 90% by weight being referred to as the high-cis type and polybutadiene having a cis content of less than 90% by weight being referred to as the low-cis type. An example of a low-cis polybutadiene is Li-BR (lithium-catalyzed butadiene rubber) having a cis content of 20% to 50% by weight. Particularly good properties and low hysteresis of the rubber mixture are achieved with a high-cis BR.

The polybutadiene(s) employed may be end group-modified with modifications and functionalizations and/or be functionalized along the polymer chains. The modification may be selected from modifications with hydroxyl groups and/or ethoxy groups and/or epoxy groups and/or siloxane groups and/or amino groups and/or aminosiloxane and/or carboxyl groups and/or phthalocyanine groups and/or silane-sulfide groups. However, further modifications known to those skilled in the art, also referred to as functionalizations, are also useful. Metal atoms may be a constituent of such functionalizations.

In the case where at least one styrene-butadiene rubber (styrene-butadiene copolymer) is present in the rubber mixture, this may be selected from solution-polymerized styrene-butadiene rubber (SSBR) and emulsion-polymerized styrene-butadiene rubber (ESBR), a mixture of at least one SSBR and at least one ESBR also being employable. The terms “styrene-butadiene rubber” and “styrene-butadiene copolymer” are used synonymously in the context of the present invention.

The styrene-butadiene copolymer used may be end group-modified and/or functionalized along the polymer chains with the modifications and functionalizations recited above for the polybutadiene.

The at least one diene rubber is preferably selected from the group consisting of natural polyisoprene (NR, natural rubber), synthetic polyisoprene (IR), butadiene rubber (BR), solution-polymerized styrene-butadiene rubber (SSBR), emulsion-polymerized styrene-butadiene rubber (ESBR), butyl rubber (IIR) and halobutyl rubber.

In a particularly preferred embodiment of the invention the at least one diene rubber is selected from the group consisting of natural polyisoprene (NR), synthetic polyisoprene (IR), butadiene rubber (BR), solution-polymerized styrene-butadiene rubber (SSBR) and emulsion-polymerized styrene-butadiene rubber (ESBR).

In a particularly advantageous embodiment of the invention the rubber mixture comprises at least one natural polyisoprene (NR) and/or synthetic polyisoprene (IR), preferably in amounts of 50 to 100 phr, and in one particularly advantageous embodiment of the invention 80 to 100 phr, very particularly preferably 95 to 100 phr, in turn preferably 100 phr. Such a rubber mixture especially shows optimized tear properties and abrasion properties coupled with good processability and reversion stability.

If the rubber mixture contains less than 100 phr of NR and/or IR it preferably contains as a further rubber at least one diene rubber selected from the group consisting of butadiene rubber (BR), solution-polymerized styrene-butadiene rubber (SSBR) and emulsion-polymerized styrene-butadiene rubber (ESBR).

In a further particularly advantageous embodiment of the invention the rubber mixture comprises at least one natural polyisoprene (NR), preferably in amounts of 5 to 55 phr, and in one particularly advantageous embodiment of the invention 5 to 25 phr, very particularly preferably 5 to 20 phr. Such a rubber mixture especially exhibits good processability and reversion stability and optimized tear properties and optimal rolling resistance characteristics.

In a further particularly advantageous embodiment of the invention the rubber mixture comprises at least one polybutadiene (BR, butadiene rubber), preferably in amounts of 10 to 80 phr, particularly preferably 10 to 50 phr, and in a particularly advantageous embodiment of the invention 15 to 40 phr. This achieves particularly good tear and abrasion properties of the rubber mixture according to the invention and optimal braking characteristics.

In a further particularly advantageous embodiment of the invention the rubber mixture comprises at least one solution-polymerized styrene-butadiene rubber (SSBR), preferably in amounts of 10 to 80 phr, particularly preferably 30 to 80 phr, and in one particularly advantageous embodiment of the invention 50 to 70 phr. This achieves particularly good rolling resistance properties of the rubber mixture according to the invention. In particularly advantageous embodiments of the invention SSBR is employed in combination with at least one further rubber to achieve an optimal and balanced profile of properties.

It is preferable when the rubber mixture contains at least one filler preferably in amounts of 30 to 500 phr, particularly preferably 50 to 400 phr, in turn preferably 80 to 300 phr.

In advantageous embodiments of the invention the filler is a reinforcing filler which is preferably selected from the group consisting of carbon blacks and silicon dioxide.

Suitable carbon blacks include any carbon black types known to those skilled in the art. It is preferable when the carbon black is selected from industrial carbon blacks and pyrolysis carbon blacks, wherein industrial carbon blacks are more preferred.

It is preferable when the carbon black has an iodine number according to ASTM D 1510, also known as the iodine adsorption number, between 30 and 250 g/kg, preferably 30 to 180 g/kg, particularly preferably 40 to 180 g/kg, and very particularly preferably 40 to 130 g/kg, and a DBP number according to ASTM D 2414 of 30 to 200 ml/100 g, preferably 70 to 200 ml/100 g, particularly preferably 90 to 200 ml/100 g.

The DBP number in accordance with ASTM D 2414 determines the specific absorption volume of a carbon black or a light-colored filler by means of dibutyl phthalate.

The use of such a type of carbon black in the rubber mixture, in particular for vehicle tires, ensures the best possible compromise between abrasion resistance and heat buildup, which in turn influences the ecologically relevant rolling resistance.

A particularly suitable and preferred carbon black is one having an iodine adsorption number between 80 and 110 g/kg and a DBP number of 100 to 130 ml/100 g, such as in particular carbon black of type N339.

The silicon dioxide is preferably amorphous silicon dioxide, for example precipitated silica, which is also referred to as precipitated silicon dioxide. However, it is alternatively also possible to employ pyrogenic silicon dioxide for example.

However, particular preference is given to using a finely divided, precipitated silica which has a nitrogen surface area (BET surface area) (in accordance with DIN ISO 9277 and DIN 66132) of 35 to 400 m²/g, preferably 35 to 350 m²/g, more preferably 85 to 320 m²/g and most preferably 120 to 235 m²/g, and a CTAB surface area (in accordance with ASTM D 3765) of 30 to 400 m²/g, preferably 30 to 330 m²/g, more preferably 80 to 300 m²/g and most preferably 115 to 200 m²/g. Such silicas lead, for example in rubber mixtures for tire treads, to particularly good physical properties of the vulcanizates. Advantages in mixture processing by way of a reduction in mixing time can also result here while retaining the same product properties, leading to improved productivity. Silicas used may thus, for example, be either those of the Ultrasil® VN3 type (trade name) from Evonik or highly dispersible silicas known as HD silicas (e.g. Zeosil® 1165 MP from Solvay).

In particularly advantageous embodiments of the invention the rubber mixture contains at least one silica as filler, preferably in amounts of 30 to 500 phr, particularly preferably 50 to 400 phr, in turn preferably 80 to 300 phr.

In these quantities silica is especially present as the sole or primary filler (more than 50% by weight based on total filler amount).

In further advantageous embodiments of the invention the rubber mixture contains at least one silica as further filler, preferably in amounts of 5 to 100 phr, particularly preferably 5 to 80 phr, in turn preferably 10 to 60 phr.

In these quantities silica is especially present as a further filler in addition to another primary filler, such as in particular a carbon black.

The terms “silicic acid” and “silica” are used synonymously in the context of the present invention.

In particularly advantageous embodiments of the invention the rubber mixture according to the invention contains 0.1 to 60 phr, preferably 3 to 40 phr, particularly preferably 5 to 30 phr, very particularly preferably 5 to 15 phr, of at least one carbon black. In these quantities carbon black is especially present as a further filler in addition to a primary filler, such as in particular silica.

In further advantageous embodiments of the invention the rubber mixture according to the invention contains 30 to 300 phr, preferably 30 to 200 phr, particularly preferably 40 to 100 phr, of at least one carbon black. In these quantities carbon black is present as the sole or primary filler and is therefore optionally present in combination with silica in the abovementioned smaller amounts.

In a particularly advantageous embodiment of the invention the rubber mixture contains 5 to 60 phr, particularly preferably 5 to 40 phr, of at least one carbon black and 50 to 300 phr, preferably 80 to 200 phr, of at least one silica.

The rubber mixture may additionally contain further fillers which are reinforcing or non-reinforcing.

Within the context of the present invention, the further (non-reinforcing) fillers include aluminosilicates, kaolin, chalk, starch, magnesium oxide, titanium dioxide, or rubber gels and also fibers (for example aramid fibers, glass fibers, carbon fibers, cellulose fibers).

Further, optionally reinforcing fillers are for example carbon nanotubes ((CNTs), including discrete CNTs, hollow carbon fibers (HCF) and modified CNTs comprising one or more functional groups such as hydroxy, carboxy and carbonyl groups), graphite and graphene and what is known as “carbon-silica dual-phase filler”.

In the context of the present invention zinc oxide is not included among the fillers.

The rubber mixture can further comprise customary additives in customary parts by weight which are added preferably in at least one primary mixing stage during the production of said mixture. These additives include
a) aging stabilizers known in the prior art,
such as for example p-phenylenediamine, such as N-phenyl-N′-(1,3-dimethylbutyl)-p-phenylenediamine (6PPD), N,N′-diphenyl-p-phenylenediamine (DPPD), N-(1-phenylethyl)-N′-phenyl-p-phenylenediamine (SPPD), N,N′-ditolyl-p-phenylenediamine (DTPD), N-(1,4-dimethylpentyl)-N′-phenyl-p-phenylenediamine (7PPD), N-isopropyl-N′-phenyl-p-phenylenediamine (IPPD),
or dihydroquinolines, such as 2,2,4-trimethyl-1,2-dihydroquinoline (TMQ),
b) activators, for example zinc oxide and fatty acids (e.g. stearic acid) and/or other activators, such as zinc complexes, for example zinc ethylhexanoate,
c) activators and/or agents for binding fillers, in particular carbon black or silica, for example S-(3-aminopropyl)thiosulfuric acid and/or metal salts thereof (bonding of carbon black) and silane coupling agents (binding to silicon dioxide, in particular silica),
d) antiozonant waxes,
e) resins, especially tackifying resins,
f) masticating aids, for example 2,2′-dibenzamidodiphenyl disulfide (DBD), and g) processing aids, such as in particular fatty acid esters and metal soaps, for example zinc soaps and/or calcium soaps,
h) plasticizers, such as in particular aromatic, naphthenic or paraffinic mineral oil plasticizers, for example MES (mild extraction solvate) or RAE (residual aromatic extract) or TDAE (treated distillate aromatic extract), or rubber-to-liquid oils (RTL) or biomass-to-liquid oils (BTL) preferably having a content of polycyclic aromatics of less than 3% by weight according to method IP 346 or triglycerides, for example rapeseed oil or factices or hydrocarbon resins or liquid polymers having a mean molecular weight (determination by GPC=gel permeation chromatography, in accordance with BS ISO 11344:2004) between 500 and 20 000 g/mol.

When using mineral oil this is preferably selected from the group consisting of DAE (distillated aromatic extracts), RAE (residual aromatic extract), TDAE (treated distillated aromatic extracts), MES (mild extracted solvents) and naphthenic oils.

In particularly advantageous embodiments the rubber mixture according to the invention contains no aging stabilizers from the group of p-phenylenediamines, in particular those listed above under a), in addition to the inventive compound of formula I), in particular of formula II) and/or III). In a particularly preferred embodiment the rubber mixture according to the invention especially contains 0 to 0.1 phr, in particular 0 phr, of further aging stabilizers based on p-phenylenediamines and selected from the group containing, preferably consisting of, N-phenyl-N′-(1,3-dimethylbutyl)-p-phenylenediamine (6PPD), N-(1-phenylethyl)-N′-phenyl-p-phenylenediamine (SPPD), N,N′-diphenyl-p-phenylenediamine (DPPD), N,N′-ditolyl-p-phenylenediamine (DTPD), N-isopropyl-N′-phenyl-p-phenylenediamine (IPPD), N-(1,4-dimethylpentyl)-N′-phenyl-p-phenylenediamine (7PPD).

The preferably very small amounts of 0 to 0.1 phr, particularly preferably 0 phr, of p-phenylenediamines, and the compound of formula I), in particular of formula II), in particular of formula III), present according to the invention makes it possible to achieve a comparable protective effect at lower toxicity. The inventive compound of formula I), in particular of formula II), in particular of formula III), replaces the recited p-phenylenediamines known in the prior art.

In further advantageous embodiments of the invention at least one further representative of the recited p-phenylenediamine aging stabilizers is present, and so the compound according to the invention only partially replaces the p-phenylendiamines known in the prior art. This also achieves the advantage according to the invention, just not to an optimal extent.

In advantageous embodiments aging stabilizers based on dihydroquinoline, such as TMQ, are present in the rubber mixture in addition to the inventive compound of formula I). The amount of dihydroquinolines present, such as especially TMQ, is preferably 0.1 to 3, in particular 0.5 to 1.5, phr.

Antiozonant waxes (group d above) are considered separately and in preferred embodiments of the invention are present in the rubber mixture irrespective of whether additional aging stabilizers a) are present.

The silane coupling agents may be any of the types known to those skilled in the art.

Furthermore, one or more different silane coupling agents may be used in combination with one another. The rubber mixture may thus contain a mixture of different silanes.

The silane coupling agents react with the surface silanol groups of the silicon dioxide, in particular of the silica, or other polar groups during the mixing of the rubber/the rubber mixture (in situ) or in the context of a pretreatment (premodification) even before addition of the filler to the rubber.

Coupling agents known from the prior art are bifunctional organosilanes having at least one alkoxy, cycloalkoxy or phenoxy group as a leaving group on the silicon atom and having as another functionality a group which, possibly after cleavage, can enter into a chemical reaction with the double bonds of the polymer. The latter group may for example comprise the following chemical groups:
—SCN, —SH, —NH₂ or —Sₓ— (with x=2 to 8).

Employable silane coupling agents thus include for example 3-mercaptopropyltriethoxysilane, 3-thiocyanatopropyltrimethoxysilane or 3,3′-bis(triethoxysilylpropyl) polysulfides having 2 to 8 sulfur atoms, for example 3,3′-bis(triethoxysilylpropyl) tetrasulfide (TESPT), the corresponding disulfide (TESPD), or else mixtures of the sulfides having 1 to 8 sulfur atoms with different contents of the various sulfides. TESPT may for example also be added as a mixture with carbon black (trade name X50S® from Evonik).

Blocked mercaptosilanes as known for example from WO 99/09036 may also be used as a silane coupling agent. It is also possible to use silanes as described in WO 2008/083241 A1, WO 2008/083242 A1, WO 2008/083243 A1 and WO 2008/083244 A1. Employable silanes include for example those marketed by Momentive, USA in a number of variants under the name NXT, such as especially 3-octanoylthio-1-propyltriethoxysilane, or those marketed by Evonik Industries under the name VP Si 363®.

The total proportion of further additives is preferably 3 to 150 phr, more preferably 3 to 100 phr and most preferably 5 to 80 phr.

Zinc oxide (ZnO) may be included in the total proportion of further additives in the abovementioned amounts.

This may be any type of zinc oxide known to those skilled in the art, for example ZnO granules or powder. The zinc oxide conventionally used generally has a BET surface area of less than 10 m²/g. However, it is also possible to use a zinc oxide having a BET surface area of 10 to 100 m²/g, for example so-called “nano zinc oxides”.

The inventive rubber mixture is preferably employed in vulcanized form, in particular in vehicle tires or other vulcanized technical rubber articles.

The terms “vulcanized” and “crosslinked” are used synonymously in the context of the present invention.

The vulcanization of the rubber mixture of the invention is preferably conducted in the presence of sulfur and/or sulfur donors with the aid of vulcanization accelerators, it being possible for some vulcanization accelerators to act simultaneously as sulfur donors. The accelerator is selected from the group consisting of thiazole accelerators, mercapto accelerators, sulfenamide accelerators, thiocarbamate accelerators, thiuram accelerators, thiophosphate accelerators, thiourea accelerators, xanthogenate accelerators and guanidine accelerators.

It is preferable to use a sulfenamide accelerator selected from the group consisting of N-cyclohexyl-2-benzothiazolesulfenamide (CBS), N,N-dicyclohexylbenzothiazole-2-sulfenamide (DCBS), benzothiazyl-2-sulfenomorpholide (MBS), N-tert-butyl-2-benzothiazylsulfenamide (TBBS) and guanidine accelerators such as diphenylguanidine (DPG).

The sulfur donor substances used may be any sulfur donor substances known to those skilled in the art.

Vulcanization retarders may also be present in the rubber mixture.

Production of the rubber mixture according to the invention is preferably otherwise carried out by the processes customary in the rubber industry comprising initially producing in one or more mixing stages a primary mixture comprising all constituents except the vulcanization system (for example sulfur and vulcanization-influencing substances). The finished mixture is produced by adding the vulcanization system in a final mixing stage.

The finished mixture is for example processed further and brought into the appropriate shape by means of an extrusion operation or calendering.

The rubber mixture according to the invention is particularly suitable for use in vehicle tires, especially pneumatic vehicle tires. Use in all tire components is conceivable in principle, in particular in an outer component, in particular and preferably in the flange profile, tread and/or sidewall. In the case of a tread with cap/base construction, the rubber mixture according to the invention is preferably used at least in the cap.

For use in vehicle tires the mixture as a finished mixture prior to vulcanization is brought into the corresponding shape, preferably of an outer component, and during production of the green vehicle tire is applied in the known manner.

Production of the rubber mixture according to the invention for use as any other body mixture in vehicle tires is carried out as described above. The difference lies in the shaping after the extrusion operation/the calendering of the mixture. The shapes thus obtained of the as-yet unvulcanized rubber mixture for one or more different body mixtures then serve for the construction of a green tire.

“Body mixture” refers here to the rubber mixtures for the inner components of a tire, such as essentially squeegee, inner liner (inner layer), core profile, belt, shoulder, belt profile, carcass, bead reinforcement, bead profile, flange profile and bandage.

The as-yet unvulcanized green tire is subsequently vulcanized.

For use of the rubber mixture of the invention in drive belts and other belts, especially in conveyor belts, the extruded, as-yet unvulcanized mixture is brought into the appropriate shape and often provided at the same time or subsequently with strength members, for example synthetic fibers or steel cords. This usually affords a multi-ply construction consisting of one and/or more plies of rubber mixture, one and/or more plies of identical and/or different strength members and one and/or more further plies of the same and/or another rubber mixture.

The present invention further provides a vehicle tire comprising the rubber mixture according to the invention containing the compound according to the invention in at least one component.

The vulcanized vehicle tire, in at least one component, comprises a vulcanizate of at least one rubber mixture of the invention. It is known to those skilled in the art that most substances, for example the rubbers present, are present or may be present in chemically modified form either already after mixing or only after vulcanization.

In the context of the present invention “vehicle tires” are to be understood as meaning pneumatic vehicle tires and solid rubber tires, including tires for industrial and construction site vehicles, truck, car and two-wheeled-vehicle tires.

It is preferable when the vehicle tire according to the invention comprises the rubber mixture according to the invention in at least one outer component, wherein the outer component is preferably a tread, a sidewall and/or a flange profile.

The vehicle tire according to the invention may accordingly contain the rubber mixture according to the invention containing the inventive compound of formula I), in particular of formula II), in particular of formula III), in a plurality of components, optionally in an adapted composition.

The invention shall now be more particularly elucidated below with reference to working examples.

The compound of formula III) as a preferred embodiment of the compound of formula I) or II) was produced as follows, as shown in formula XI):

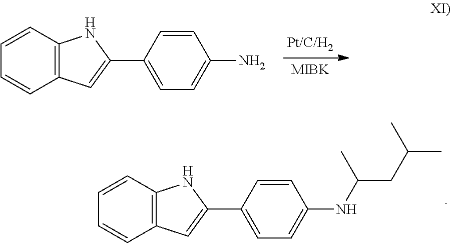 [SMILES: Nc1ccc(-c2cc3ccccc3[nH]2)cc1 ; CC(C)CC(C)Nc1ccc(-c2cc3ccccc3[nH]2)cc1]

5.40 g (25.9 mmol, 1 eq) of 2-(4-aminophenyl)-1H-indole, 1.09 g of platinum on carbon (Pt/C) (5%) (0.2 g on 4.67 mmol of substrate) and 50.0 ml of methyl isobutyl ketone (MIBK) were weighed into a stainless steel autoclave fitted with a Teflon liner. The reaction mixture was subsequently subjected to hydrogen at a pressure of 20 bar and stirred for 10 hours at 60° C. Upon termination of the reaction the excess hydrogen was released and the suspension was filtered through Celite® and washed with ethanol. The filtrate was evaporated to dryness and dried under vacuum. It was recrystallized from cyclohexane. A grayish to violet solid was obtained; yield 5.40 g (710% of theory).

¹H-NMR (nuclear magnetic resonance) (500 MHz, DMSO-d6) δ=11.16 (s, 1H), 7.56 (d, J=8.6 Hz, 2H), 7.43 (dd, J=7.7, 1.1 Hz, 1H), 7.32 (dd, J=7.9, 1.0 Hz, 1H), 7.00 (ddd, J=8.1, 7.0, 1.2 Hz, 1H), 6.93 (ddd, J=8.0, 7.0, 1.1 Hz, 1H), 6.63 (d, J=8.6 Hz, 2H), 6.57 (d, J=1.4 Hz, 1H), 5.56 (d, J=8.5 Hz, 1H), 3.56-3.47 (m, 1H), 1.74 (dt, J=13.5, 6.7 Hz, 1H), 1.47 (dt, J=13.9, 7.1 Hz, 1H), 1.25 (dt, J=13.5, 6.9 Hz, 1H), 1.11 (d, J=6.2 Hz, 3H), 0.93 (d, J=6.6 Hz, 3H), 0.89 (d, J=6.6 Hz, 3H).

¹³C-NMR (126 MHz, DMSO-d6) δ=148.5, 139.6, 137.1, 129.6, 126.6, 120.7, 119.7, 119.5, 119.4, 112.7, 111.2, 95.8, 46.4, 45.8, 26.8, 25.0, 23.2, 23.1, 21.2.

ESI-MS (electrospray ionization mass spectrometry) [M+H]⁺=293.

Melting point: 142° C.

### Measurement of Oxidation Induction Time (OIT)

The compound of formula III) was investigated under laboratory conditions for their potential protective effect as aging stabilizers by measurement of the oxidation induction time.

To this end the compounds of formula III) and 6-PPD in each case together with a polymer (liquid synthetic polyisoprene (IR), LIR-50, Kuraray, weight average molecular weight distribution M_{W}=54 000 g/mol, glass transition temperature T_{g}=−63° C.) were heated at constant temperature (180° C.) until onset of oxidation (start temperature 35° C., heating to 170° C. at a heating rate of 20 K/min (kelvin per minute), heating to 180° C. at a heating rate of 1 K/min; purge gas: nitrogen (N₂), volume flow 50 mL/min). The specimen was isothermally maintained for 5 minutes at 180° C. under an N₂ atmosphere and the atmosphere was then switched to an O₂ atmosphere (volume flow 50 mL/min).

Oxidation was determined via a peak using DSC (differential scanning calorimetry).

The time in minutes to oxidation was measured.

The results compared to known aging stabilizer 6-PPD are summarized in table 1.

TABLE 1

| Substance | [min] |
|---|---|
| 6-PPD | 116 ± 10 |
| 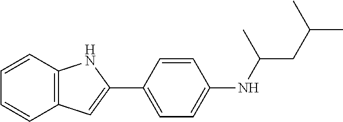 [SMILES: CC(C)CC(C)Nc1ccc(-c2cc3ccccc3[nH]2)cc1] | 121 ± 10 |

Taking into account the measurement accuracy of (plus/minus) 10 minutes it is apparent that the compound of formula III) is an adequate substitute for the more health-hazardous compound 6-PPD.

For use in a rubber mixture for vehicle tires the inventive compound of formula I), for example of formula III), is added in one of the mixing stages during production of the rubber mixture in a manner known to those skilled in the art for example instead of the aging stabilizers known to those skilled in the art, such as 6PPD, 7PPD or IPPD etc.

Accordingly the compound of formula III) was incorporated into an exemplary rubber mixture according to the invention as shown in table 2. The resulting inventive example is labelled E1.

Serving as a comparison is a rubber mixture V1 containing 6PPD instead of the compound of formula III) as aging stabilizer with the remaining composition being identical. The amounts in table 2 are expressed in units of phr.

The mixture was produced according to the process customary in the rubber industry under standard conditions in three stages in a laboratory mixer having a volume of 300 milliliters to 3 liters wherein initially in the first mixing stage (preliminary mixing stage) all constituents apart from the vulcanization system (sulfur and vulcanization influencers) were mixed at 145° C. to 165° C., target temperatures of 152° C. to 157° C., for 200 to 600 seconds.

In the second stage the mixture from the first stage was mixed once again. Addition of the vulcanization system in the third stage (final mixing stage) afforded the final mixture, mixing being carried out at 90° C. to 120° C. for 180 to 300 seconds.

Test specimens were produced from all mixtures by vulcanization after t95 to t100 (measured using a moving die rheometer according to ASTM D 5289-12/ISO 6502) under pressure at 160° C. to 170° C.

In addition, a portion of the test specimens of both V1 and E1 were aged (70° C. for 28 days in air).

The following material properties typical for the rubber industry were determined for all test specimens:
Rebound resilience at room temperature (RT) according to ISO 4662 or ASTM D 1054
Stress value at 300% elongation (M 300) and breaking elongation at room temperature (RT) according to DIN 53 504

For V1 and E1 the difference between the values for the un-aged and aged samples was determined.

The obtained values for V1 were in each case normalized to 100% for reference.

The values obtained for E1 (difference between un-aged and aged) are reported relative to this respective V1 reference as performance %, wherein values above 100% are advantageous

As is apparent from table 2 the inventive compound of formula III) as a representative of the compound of formula I) results in improved aging stabilization since important properties such as stress value at 300% elongation (300 modulus), breaking elongation and rebound elasticity are in each case at a higher level for E1 than for V1 after aging.

TABLE 2

| | V1 | E1 |
|---|---|---|
| Constituent | | |
| IR | 100 | 100 |
| Carbon black N 339 | 50 | 50 |
| MES | 8 | 8 |
| 6PPD | 2 | — |
| Compound of formula III) | — | 2 |
| ZnO | 3 | 3 |
| Stearic acid | 2 | 2 |
| TBBS | 1.2 | 1.2 |
| Sulfur | 1.2 | 1.2 |
| Properties | | |
| M300 | 100 | 128 |
| Elongation at break | 100 | 105 |
| Rebound resilience RT | 100 | 106 |

1.-15. (canceled)

16. A compound of formula I): 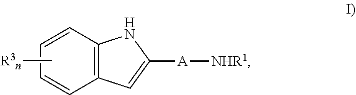
wherein A is an aromatic radical optionally bearing additional substituents, and wherein R¹ is selected from the group consisting of
xi) aromatic radicals, wherein the aromatic radicals optionally bear substituents selected from the group consisting of halogen radicals, cyano radicals, ester radicals, ketone radicals, ether radicals and thioether radicals,
and xii) linear, branched and cyclic aliphatic C₁- to C₁₂-radicals,
and xiii) combinations of aromatic and aliphatic C₁- to C₁₂-radicals, and wherein R³ is selected from the group consisting of linear, branched and cyclic, saturated and unsaturated, aliphatic C₁- to C₁₂-radicals optionally bearing one or more halogen substituents, aryl radicals optionally bearing one or more halogen substituents, and halogen radicals, cyano radicals, ester radicals, ketone radicals, ether radicals and thioether radicals, and wherein n assumes the value 0 or 1 or 2 or 3 or 4, wherein when n is 2 or 3 or 4 the radicals R³ are independently of one another identical or different.

17. The compound as claimed in claim 16, wherein A is selected from the group consisting of phenylene, naphthylene and antracenylene radicals, and phenylene, naphthylene and anthracenylene radicals, and aryl radicals, cyano radicals, halogen radicals, ether radicals and thioether radicals.

18. The compound of claim 17, wherein the anthracenylene radicals have one or more substituents selected from the group consisting of linear, branched and cyclic aliphatic C1- to C12-radicals.

19. The compound of claim 17, wherein the halogen radicals include fluorine, bromine or chlorine.

20. The compound as claimed in claim 16, wherein the compound has the structure of formula II): 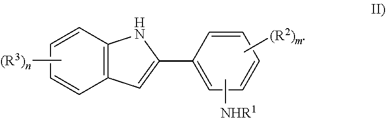
wherein R¹ is selected from the group consisting of
xi) aromatic radicals, wherein the aromatic radicals optionally bear substituents selected from the group consisting of halogen radicals, cyano radicals, ester radicals, ketone radicals, ether radicals and thioether radicals,
and xii) linear, branched and cyclic aliphatic C₁- to C₁₂-radicals,
and xiii) combinations of aromatic and aliphatic C₁- to C₁₂-radicals, and wherein R² is selected from the group consisting of linear, branched and cyclic, saturated and unsaturated, aliphatic C₁- to C₁₂-radicals optionally bearing one or more halogen substituents, aryl radicals optionally bearing one or more halogen substituents, and halogen radicals, cyano radicals, ester radicals, ketone radicals, ether radicals and thioether radicals, and
wherein m assumes the value 0 or 1 or 2 or 3 or 4, wherein when m is 2 or 3 or 4 the radicals R² are independently of one another identical or different, and wherein R³ is selected from the group consisting of linear, branched and cyclic, saturated and unsaturated, aliphatic C₁- to C₁₂-radicals optionally bearing one or more halogen substituents, aryl radicals optionally bearing one or more halogen substituents and halogen radicals, cyano radicals, ester radicals, ketone radicals, ether radicals and thioether radicals and wherein n assumes the value 0 or 1 or 2 or 3 or 4, wherein when n is 2 or 3 or 4 the radicals R³ are independently of one another identical or different.

21. The compound as claimed in claim 16, wherein n is 0 (zero).

22. The compound as claimed in claim 16, wherein R¹ is bonded to the nitrogen atom (N) via a tertiary carbon atom.

23. The compound as claimed in claim 16, wherein R¹ is a branched alkyl radical having 3 to 12 carbon atoms.

24. The compound as claimed in claim 16, wherein R¹ is a branched alkyl radical having 3 to 8 carbon atoms.

25. The compound as claimed in claim 16, wherein R¹ is selected from 1,3-dimethylbutyl and cyclohexyl radicals.

26. The compound as claimed in claim 16, wherein R¹ is a 1,3-dimethylbutyl radical.

27. The compound as claimed in claim 20, wherein m is 0 (zero).

28. The compound as claimed in claim 16, wherein the compound has the structure of formula III):

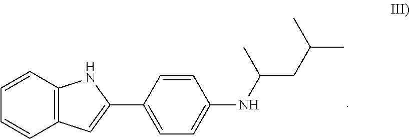 [SMILES: CC(C)CC(C)Nc1ccc(-c2cc3ccccc3[nH]2)cc1]

29. A method of using the compound of claim 16, comprising:
providing the compound, and
using the compound as an aging stabilizer in a vehicle tire and/or a technical rubber article and/or oil and/or lubricant.

30. The method of claim 29, wherein the technical rubber article is an air spring, bellows, conveyor belt, belt, drive belt, hose, rubber band, profile, a seal, a membrane, tactile sensors for medical applications or robotics applications, or a shoe sole or parts thereof.

31. A method of using the compound of claim 16, comprising:
providing the compound, and
using the compound as a dye in fibers and/or polymers and/or paper and/or in paints and/or coatings.

32. A process for producing the compound of formula I) which comprises the following process steps:
a) providing the compound of formula A) 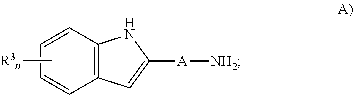
b) reacting the compound of formula A) with hydrogen or a hydrogenating reagent and a ketone or aldehyde to afford the compound of formula I) 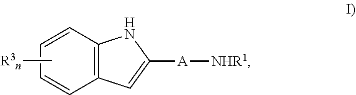
wherein A is is an aromatic radical optionally bearing additional substituents, and
wherein R¹ is selected from the group consisting of
xi) aromatic radicals, wherein the aromatic radicals optionally bear substituents selected from the group consisting of halogen radicals, cyano radicals, ester radicals, ketone radicals, ether radicals and thioether radicals,
and xii) linear, branched and cyclic aliphatic C₁- to C₁₂-radicals,
and xiii) combinations of aromatic and aliphatic C₁- to C₁₂-radicals, and wherein R³ is selected from the group consisting of linear, branched and cyclic, saturated and unsaturated, aliphatic C₁- to C₁₂-radicals optionally bearing one or more halogen substituents, aryl radicals optionally bearing one or more halogen substituents and halogen radicals, wcyano radicals, ester radicals, ketone radicals, ether radicals and thioether radicals and wherein n assumes the value 0 or 1 or 2 or 3 or 4, wherein when n is 2 or 3 or 4 the radicals R³ are independently of one another identical or different.

33. The process as claimed in claim 32, wherein in step b) the reaction with hydrogen (H₂) and the ketone or aldehyde is carried out using a hydrogenation catalyst and at a temperature of 50° C. to 70° C. and the reaction mixture is subjected to hydrogen at a pressure of 15 to 25 bar and the reaction is carried out in an autoclave or in another pressure reactor.

34. A rubber mixture containing the compound as claimed in claim 16, wherein the rubber mixture contains at least one diene rubber which is selected from the group consisting of natural polyisoprene (NR rubber), synthetic polyisoprene (IR), butadiene rubber (BR), solution-polymerized styrene-butadiene rubber (SSBR), emulsion-polymerized styrene-butadiene rubber (ESBR), butyl rubber (IIR) and halobutyl rubber.

35. A vehicle tire comprising the rubber mixture as claimed in claim 34 in
at least one component.
